(19) United States
(12) Patent Application Publication
(10) Pub. No.: US 2004/0126341 A1
(43) Pub. Date: Jul. 1, 2004

(54) Process for protecting and modulating tight junctions

(76) Inventors: Gilles Pauly, Nancy (FR); Christine Jeanmaire, Nancy (FR)

Correspondence Address: COGNIS CORPORATION, PATENT DEPARTMENT, 300 BROOKSIDE AVENUE, AMBLER, PA, 19002, US

(21) Appl. No.: 10/625,153

(22) Filed: Jul. 23, 2003

(30) Foreign Application Priority Data
Jul. 25, 2002 (EP) 02291883.3

(51) Int. Cl. A61K 7/42; A61K 7/06; A61K 7/11

(52) U.S. Cl. 424/59; 424/70.14

(57) ABSTRACT

The invention relates to a cosmetic treatment process for improving the epidermal barrier function of the skin, scalp and mucous membrane, characterized in that a preparation containing at least one substance which modulates tight junctions and/or which modulates occludin and/or claudins and/or which modulates the Zona occludens through the proteins ZO-1, ZO-2 and/or ZO-3 is topically applied. The present invention also relates to the use of a substance which modulates tight junctions and/or which modulates occludin and/or claudins and/or which modulates the Zona occludens through the proteins ZO-1, ZO-2 and/or ZO-3 for the production of cosmetic preparations for improving the epidermal barrier function of the skin, scalp and mucous membrane, to the use of this substance for the production of cosmetic preparations for protection against aging of the skin and to the use of the substance for the production of cosmetic preparations for protection against the harmful effects of environmental toxins and UV radiation.

### BACKGROUND OF THE INVENTION

Tight junctions (also known as occluding junctions or Zonula occuladens) connect adjacent cells of a tissue to one another at their luminal ends. They separate the liquid spaces on the luminal and serosal surface of the epithelium from one another and form a boundary between the apical membrane (cell side to the lumen) and the basolateral membrane (cell body and base) of individual cells.

Tight junctions perform two key functions. They prevent the passage of molecules and ions through the interstices of cells and thus protect cells against harmful substances because materials that have to pass through the cell by diffusion or active transport are taken up under control. They block the exchange or the flow of intracellular substances, such as membrane proteins for example, and thus contribute to the maintenance of special functions, such as receptor-activated endocytosis or exocytosis on the basolateral surface. Tight junctions thus also facilitate the important maintenance of concentration gradients over epithelia.

Tight junctions consist of crosslinked protein strands anchored in the membrane. The main components are proteins, such as occludin—a transmembranal protein of 65 kDa, SO-1 (Zona occludens-1), ZO-2, ZO-3 and the group of claudins of which there are more than 20 different proteins.

For a long time, it was assumed that tight junctions were only present in simple tissues, for example in the epithelium which delimits blood vessels. Since then, however, proteins, such as occludin, ZO-1 and ZO-2, which have to be associated with tight junctions, have also been found in the skin.

Using a group of mice that had died with dry, wrinkled skin, M. Furuse et al. were able to show that the epidermal barrier function of the skin had been seriously damaged by the absence of claudins in the tight junctions of the Stratum granulosum so that the cells were unable to hold any water [M. Furuse, M. Hata, K. Furuse et al., The Journal of Cell Biology, 156 (No. 6): 1099-1111, 2002].

Malfunctions of the tight junctions are also involved in the development of certain diseases, such as inflammatory bowel disease (IBD) and psoriasis. In studies of psoriasis, clear differences in the location of the proteins between healthy and pathological skin were found in the tight junctions by immunohistological determinations with antibodies against proteins.

International patent application WO 99/35166 discloses cyclic peptides which influence the occludin-controlled cell adhesion and pharmaceutical preparations containing these peptides. The document in question describes inter alia methods for increasing vasopermeability, for reducing cell adhesion, for improving the absorption of medicaments, particularly cytostatic agents, or taking up medicaments into the central nervous system and a method for reducing angiogenesis which use this mechanism for modulating occludin-controlled cell adhesion. A process for detecting occludin-controlled cells is also described.

The problem addressed by the present invention was to provide new mechanisms for improving the barrier function of the skin, scalp and mucous membrane which would contribute towards retarding aging of the skin and towards protecting the skin, scalp and mucous membrane against environmental influences, such as toxic substances or UV radiation, and hence could be effectively used in cosmetic preparations for dermal application.

### SUMMARY OF THE INVENTION

This invention relates generally to cosmetic preparations and, more particularly, to a cosmetic process for improving the epidermal barrier function of the skin, scalp and mucous membrane and for protecting human skin against aging and against the harmful effects of environmental toxins and UV radiation. The invention also relates to the use of a substance which modulates tight junctions and/or which modulates occludin and/or claudins and/or which modulates the Zona occludens through the proteins ZO-1, ZO-2 and/or ZO-3 for the production of cosmetic preparations for improving the epidermal barrier function of the skin, scalp and mucous membrane and for protection against aging of the skin, toxic environmental influences and UV radiation.

The present invention relates to cosmetic treatment processes for improving the epidermal barrier function of the skin, scalp and mucous membrane, characterized in that a preparation containing at least one substance which modulates tight junctions and/or which modulates occludin and/or claudins and/or which modulates the Zona occludens through the proteins ZO-1, ZO-2 and/or ZO-3 is topically applied.

The present invention also relates to the use of a substance which modulates tight junctions and/or which modulates occludin and/or claudins and/or which modulates the Zona occludens through the proteins ZO-1, ZO-2 and/or ZO-3 for the production of cosmetic preparations for improving the epidermal barrier function of the skin, scalp and mucous membrane, to the use of this substance for the production of cosmetic preparations for protection against aging of the skin and to the use of the substance for the production of cosmetic preparations for protection against the harmful effects of environmental toxins and UV radiation.

It has surprisingly been found that the modulation of tight junctions, particularly through the proteins of the tight junctions, leads to maintenance and improvement of the epidermal barrier function of the skin, scalp and mucous membrane. This function is an essential precondition not only for health, but also for cosmetology. The topical application of a preparation containing a substance which modulates tight junctions to the skin, mucous membrane or scalp, preferably the skin and especially dry or aging skin, results in a reduced loss of moisture through the improved barrier function. In this way, it protects the skin not only against aging, but also against the harmful effect of UV radiation. In addition, a better barrier contributes to environmental toxins not being taken up into the cells.

### DEATILED DESCRIPTION OF THE INVENTION

Accordingly, the topically applied cosmetic preparations containing at least one substance which modulates tight junctions have a preventive effect against aging of the skin and against the harmful effects of environmental toxins and UV radiation.

However, the modulation of the tight junctions also leads to an improvement in the epidermal barrier function after damage already done and thus gives the skin, scalp and mucous membrane the opportunity to regenerate more quickly.

The tight junctions are preferably modulated by modulation of the proteins occludin and/or via claudins and/or via the proteins ZO-1, ZO-2 and/or ZO-3. Peptides or plant extracts may be used for this purpose.

The tight junctions may also be modulated through at least one substance selected from the group consisting of differentiation modulators, calcium modulators, growth factors, such as EGF (epidermal growth factor), TNF alpha (tumor necrosis factor alpha), cytokinins, such as interferon gamma, transcription factors, such as KLF4, vitamins, for example vitamin A and vitamin D, hormones, such as PTHrP (parathyroid hormone-related peptide), transglutaminases and caspases. Among the differentiation modulators, calcium modulators are particularly preferred.

Besides these substances, peptides or plant extracts, the cosmetic preparations may also contain UV protection factors and/or antioxidants. The combination of substances which modulate tight junctions and/or which modulate occludin and/or claudins and/or which modulate the Zona occludens through the proteins ZO-1, ZO-2 and/or ZO-3 with UV protection factors and/or antioxidants leads through the different mechanisms to a synergistic mode of action and affords excellent protection against harmful influences and aging of the skin by UV radiation.

UV Protection Factors and Antioxidants

UV protection factors in the context of the invention are, for example, organic substances (light filters) which are liquid or crystalline at room temperature and which are capable of absorbing ultraviolet radiation and of releasing the energy absorbed in the form of longer-wave radiation, for example heat. UV-B filters can be oil-soluble or water-soluble. The following are examples of oil-soluble substances: 3-benzylidene camphor or 3-benzylidene norcamphor and derivatives thereof, for example 3-(4-methylbenzylidene)-camphor; 4-aminobenzoic acid derivatives, preferably 4-(dimethylamino)-benzoic acid-2-ethylhexyl ester, 4-(dimethylamino)-benzoic acid-2-octyl ester and 4-(dimethylamino)-benzoic acid amyl ester; esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (Octocrylene); esters of salicylic acid, preferably salicylic acid-2-ethylhexyl ester, salicylic acid-4-isopropylbenzyl ester, salicylic acid homomenthyl ester; derivatives of benzophenone, preferably 2-hydroxy-4-methoxybenzophenone, 2-hydroxy-4-methoxy-4′-methylbenzophenone, 2,2′-dihydroxy-4-methoxybenzophenone; esters of benzalmalonic acid, preferably 4-methoxybenzalmalonic acid di-2-ethylhexyl ester; triazine derivatives such as, for example, 2,4,6-trianilino-(p-carbo-2′-ethyl-1′-hexyloxy)-1,3,5-triazine and Octyl Triazone or Dioctyl Butamido Triazone (Uvasorb® HEB); propane-1,3-diones such as, for example, 1-(4-tert.butylphenyl)-3-(4′-methoxyphenyl)-propane-1,3-dione; ketotricyclo(5.2.1.0)decane derivatives.

Suitable water-soluble substances are: 2-phenylbenzimidazole-5-sulfonic acid and alkali metal, alkaline earth metal, ammonium, alkylammonium, alkanolammonium and glucammonium salts thereof; sulfonic acid derivatives of benzophenones, preferably 2-hydroxy-4-methoxybenzophenone-5-sulfonic acid and salts thereof; sulfonic acid derivatives of 3-benzylidene camphor such as, for example, 4-(2-oxo-3-bornylidenemethyl)-benzene sulfonic acid and 2-methyl-5-(2-oxo-3-bornylidene)-sulfonic acid and salts thereof.

Typical UV-A filters are, in particular, derivatives of benzoyl methane such as, for example, 1-(4′-tert.butylphenyl)-3-(4′-methoxyphenyl)-propane-1,3-dione, 4-tert.butyl-4′-methoxydibenzoyl methane (Parsol 1789) or 1-phenyl-3-(4′-isopropylphenyl)-propane-1,3-dione and enamine compounds. The UV-A and UV-B filters may of course also be used in the form of mixtures. Particularly favorable combinations consist of the derivatives of benzoyl methane, for example 4-tert.butyl-4′-methoxydibenzoylmethane (Parsol® 1789) and 2-cyano-3,3-phenylcinnamic acid-2-ethyl hexyl ester (Octocrylene) in combination with esters of cinnamic acid, preferably 4-methoxycinnamic acid-2-ethyl hexyl ester and/or 4-methoxycinnamic acid propyl ester and/or 4-methoxycinnamic acid isoamyl ester. Combinations such as these are advantageously combined with water-soluble filters such as, for example, 2-phenylbenzimidazole-5-sulfonic acid and alkali metal, alkaline earth metal, ammonium, alkylammonium, alkanolammonium and glucammonium salts thereof.

Besides the soluble substances mentioned, insoluble light-blocking pigments, i.e. finely dispersed metal oxides or salts, may also be used for this purpose. Examples of suitable metal oxides are, in particular, zinc oxide and titanium dioxide and also oxides of iron, zirconium oxide, silicon, manganese, aluminum and cerium and mixtures thereof. Silicates (talcum), barium sulfate and zinc stearate may be used as salts. The oxides and salts are used in the form of the pigments for skin-care and skin-protecting emulsions and decorative cosmetics. The particles should have a mean diameter of less than 100 nm, preferably between 5 and 50 nm and more preferably between 15 and 30 nm. They may be spherical in shape although ellipsoidal particles or other non-spherical particles may also be used. The pigments may also be surface-treated, i.e. hydrophilicized or hydrophobicized. Typical examples are coated titanium dioxides, for example Titandioxid T 805 (Degussa) and Eusolex® T2000 (Merck). Suitable hydrophobic coating materials are, above all, silicones and, among these, especially trialkoxyoctylsilanes or simethicones. So-called micro- or nanopigments are preferably used in sun protection products. Micronized zinc oxide is preferably used.

Besides the two groups of primary sun protection factors mentioned above, secondary sun protection factors of the antioxidant type may also be used. Secondary sun protection factors of the antioxidant type interrupt the photochemical reaction chain which is initiated when UV rays penetrate into the skin. Typical examples are amino acids (for example glycine, histidine, tyrosine, tryptophane) and derivatives thereof, imidazoles (for example urocanic acid) and derivatives thereof, peptides, such as D,L-camosine, D-camosine, L-camosine and derivatives thereof (for example anserine), carotinoids, carotenes (for example α-carotene, β-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages (for example pmol to μmol/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, camosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, superoxide dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A cosmetic treatment process for improving the epidermal barrier function of the skin, scalp and mucous membrane, the process comprising providing a substrate to be treated, and topically applying a preparation containing at least one substance which modulates tight junctions.

2. A cosmetic treatment process for improving the epidermal barrier function of the skin, scalp and mucous membrane, the process comprising providing a substrate to be treated, and topically applying a preparation containing at least one substance which modulates occludin and/or claudins.

3. A cosmetic treatment process for improving the epidermal barrier function of the skin, scalp and mucous membrane, the process comprising providing a substrate to be treated, and topically applying a preparation containing at least one substance which modulates the Zona occludens through the proteins ZO-1, ZO-2 and/or ZO-3.

4. A cosmetic preparation comprising: (a) at least one substance selected from the group consisting of compounds which modulate tight junctions, compounds which modulate occludin and/or claudins, compounds which modulate the Zona occludens through the proteins ZO-1, ZO-2 and/or ZO-3; and (b) a compound selected from the group consisting of UV protection factors and antioxidants.
